(19) United States
(12) Patent Application Publication
(10) Pub. No.: US 2019/0262197 A1
(43) Pub. Date: Aug. 29, 2019

(54) WEARABLE ABSORBENT DEVICE AND WET WIPE COMBINATION AND METHOD

(71) Applicant: Douglas Stewart, Las Vegas, NV (US)

(72) Inventor: Douglas Stewart, Las Vegas, NV (US)

(21) Appl. No.: 15/904,360

(22) Filed: Feb. 24, 2018

(51) Int. Cl. A61F 13/84; A61F 13/15

(52) U.S. Cl. CPC A61F 13/84; A61F 13/5519; A61F 13/15585

(57) ABSTRACT

The wearable diaper and wet wipe combination disclosed herein may comprise, at least, a diaper, a storage pocket, and a plurality of wet wipes. Such a combination is advantageous to caregivers of diaper wearers requiring multiple daily changes, as the combination reduces the effective cost and difficulty of acquiring and keeping disposable diapers and wet wipes within proximity of each other. In a preferred embodiment the wearable diaper and wet wipe combination comprises three individually-packaged wet wipes enclosed in a pocket created by an affixed layer of material. Such pocket is located on the rear face of a disposable diaper and may be removably sealed so as to facilitate the storage of wet wipes and access to them when needed. A preferred system for manufacturing the present combination may involve the use of proprietary robotic arms for the manipulation and assembly of each component.

### PRIORITY NOTICE

The present application makes no claims of priority under 35 U.S.C. § 119(e) to any U.S. Provisional Patent Applications.

### CROSS REFERENCE TO RELATED PATENT APPLICATIONS

The present application makes no reference to any other related filed patent applications.

### STATEMENT REGARDING FEDERAL SPONSORSHIP

No part of this invention was a result of any federally sponsored research.

### TECHNICAL FIELD OF THE INVENTION

The present invention relates in general to wearable absorbent devices, and, more specifically, to a wearable absorbent device and wet wipe combination comprising, at least, a wearable absorbent device, a storage pocket, and a plurality of wet wipes.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

A diaper or a feminine hygiene pad is a type of wearable device that absorbs or contains waste products discharged by a wearer, preventing contamination of outer clothing or the external environment. Diapers are commonly used on infants and toddlers not yet potty trained or on adults suffering from incontinence. Feminine hygiene pads are commonly worn by females during a menstrual cycle. While such devices prevent external contamination the waste products contained by them inevitably soils the wearer, and it is strongly advised that such wearer be cleaned prior to wearing a fresh device. One common method of cleaning, especially on a wearer whose diaper is changed multiple times per day, is to use wet wipes for the removal of such soiling. A wet wipe is a moistened disposable cloth often available individually wrapped and containing antibacterial or antiseptic solutions. Baby wipes, a specific type of wet wipes, tend to comprise solutions suitable for the more sensitive skin of infants and toddlers.

It is known to have a diaper comprising absorbent polymers, resealable tapes, and elasticized waist bands which may be used once and disposed when soiled. Such a diaper may be designed with multiple layers, some of which may be cloth or non-absorbent material, and which may direct waste products towards absorbent layers. Such diapers may further comprise a wetness indicator such as a chemical that changes colors in the presence of moisture, or may comprise fragrances or essential oils that may mask the scent of the soiled diaper. Such a design may be called a “disposable diaper,” and may allow for frequent diaper changes without the need for cleaning the soiled diapers.

It is known to have a diaper comprising natural fibers or synthetic materials, or some combination thereof, which may be reusable. Such a diaper may be designed with multiple layers, some of which may be cloth or non-absorbent material, and which may direct waste products towards absorbent layers. Such diapers may specifically comprise wool, bamboo, hemp, microfiber, or polyester, and may be designed to withstand damage from moisture. The use of natural materials may further permit a reduced incidence of skin reactions in certain users. Such a design may be called a “reusable diaper,” and may allow for reduced overall diaper cost while requiring that the diapers be cleaned between uses.

It is known to have a wet wipe comprising cleansing fluids or alcohol-based ingredients, which may be made available in a multi-pack or individually-sealed. Such wet wipes may comprise a disposable paper or fiber, and may be suitable for one-time use in the cleaning of a soiled user. Such wet wipes may be napkin-sized, and may further comprise fragrances or scented oils that may mask the scent of waste products. Such a design may be called a “baby wipe,” and may be used during regular diaper changes to clean a user without the need for a bath or a wash.

There is a need in the art for a wearable absorbent device and wet wipe combination comprising, at least, a wearable absorbent device, a storage pocket, and a plurality of wet wipes. Such a device may facilitate the cleaning and changing of a diaper or feminine hygiene pad user by keeping wet wipes within the proximity of the user while the device is being worn, and may reduce the overall cost of purchasing disposable diapers or feminine hygiene pads and wet wipes by providing both items within a single device. Such a device may further reduce the incidence of failing to clean a soiled user during a changing, as the wet wipes are within the proximity of and readily accessible to the person changing the wearable absorbent device, and may be made available in a plurality of sizes so as to be wearable by users of different ages and body types.

It is to these ends that the present invention has been developed.

### BRIEF SUMMARY OF THE INVENTION

To minimize the limitations in the prior art, and to minimize other limitations that will be apparent upon reading and understanding the present specification, the present invention describes a wearable absorbent device and wet wipe combination comprising, at least, a wearable absorbent device, a storage pocket, and a plurality of wet wipes.

It is an objective of the present invention to provide a wearable absorbent device and wet wipe combination that may comprise a disposable diaper.

It is another objective of the present invention to provide a wearable absorbent device and wet wipe combination that may comprise a reusable diaper.

It is an objective of the present invention to provide a wearable absorbent device and wet wipe combination that may comprise a feminine hygiene pad.

It is another objective of the present invention to provide a wearable absorbent device and wet wipe combination that may comprise a pocket attached to the wearable absorbent device.

It is another objective of the present invention to provide a wearable absorbent device and wet wipe combination that may comprise a pocket removably attached to the wearable absorbent device.

It is another objective of the present invention to provide a wearable absorbent device and wet wipe combination that may comprise a plurality of wet wipes.

It is another objective of the present invention to provide a wearable absorbent device and wet wipe combination that may comprise a plurality of baby wipes.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates a sealed individually-wrapped disposable wet wipe packet.

FIG. 2 illustrates an opened individually-wrapped disposable wet wipe packet.

FIG. 3 illustrates a wet wipe pocket layer.

FIG. 4 illustrates a branded wet wipe pocket layer.

FIG. 5 illustrates a first embodiment of a wearable absorbent device and wet wipe combination.

FIG. 6 illustrates a first embodiment of a wearable absorbent device and wet wipe combination.

FIG. 7 illustrates a second embodiment of a wearable absorbent device and wet wipe combination.

FIG. 8 illustrates a robotic arm for placing wet wipes and installing a wet wipe pocket on a wearable absorbent device.

FIG. 9 illustrates a system for placing wet wipes and installing a wet wipe pocket on a wearable absorbent device.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

The present invention relates generally to wearable absorbent devices, and, more specifically, to a wearable absorbent device and wet wipe combination comprising, at least, a wearable absorbent device, a storage pocket, and a plurality of wet wipes.

In a first embodiment of the present invention, the wearable absorbent device may be any type of appropriate diaper, whether disposable or reusable, but is preferably a disposable diaper. Use of a disposable diaper is advantageous to the current combination, as it is contemplated that the diaper may be used once and the pre-loaded wet wipes may be used once, and then the entire combination may be disposed of to eliminate the need for cleaning or reloading the combination. The size of the diaper may be any size appropriate for use on an infant, toddler, child, or adult, but is preferably sized for use on an infant or toddler. Such a size is advantageous as infants and toddlers tend to require multiple daily diaper changes, and the combination would permit such diaper changes, and their associated cleanings, without the need for acquiring and using third-party wet wipes. A sizing appropriate for children or for adults may also be advantageous for a user not yet potty trained or suffering from incontinence. Such user may also require multiple daily diaper changes and cleanings, and so the combination would provide the same benefits to such a user.

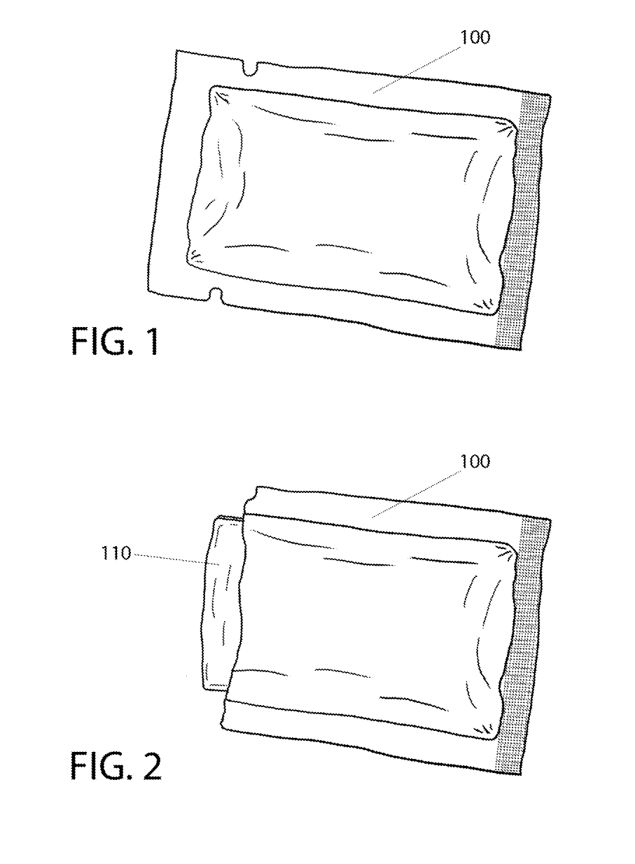

FIG. 1 illustrates a sealed individually-wrapped disposable wet wipe packet 100. The wearable absorbent device and wet wipe combination is achieved by placing a plurality of wet wipes against an outer surface of a wearable absorbent device and attaching an outer pocket layer to the wearable absorbent device in such a way as to enclose and secure the wet wipes to the wearable absorbent device. The number of wet wipes included in the combination may vary by application, but is considered to be at least one. Based on consumer feedback it is considered advantageous to package between two and ten wet wipes in the combination, and preferable to include three individually packaged wet wipes in the combination.

FIG. 2 illustrates an opened individually-wrapped disposable wet wipe packet 100 and a wet wipe 110. The plurality of wet wipes may be individually packaged or may be bulk packaged. Bulk packaging of the wet wipes may allow for a larger number of wipes to be included in the combination, and may reduce the effective cost of manufacturing the combination. Individually packaging the wet wipes may allow for the wipes to remain independently sterile and may allow a user to save excess wipes that do not get used during the cleaning process.

The figure further illustrates the relative positioning of the wet wipe 110 within the wet wipe packet 100, and visually demonstrates that a user may access the wet wipe 110 by tearing an end from the wet wipe packet 100. Such end of the wet wipe packet 100 may be defined by a plurality of notches, which may facilitate the tearing off of such end. The wet wipe 110 may then be withdrawn from the wet wipe packet 100 and used for cleaning a diaper wearer. Once used, such wet wipe 100 and wet wipe packet 110 may be disposed.

The wet wipe 100 may comprise any of a combination of cleaning and sterilizing solutions known in the art. In a preferred embodiment, the wet wipe 100 comprises a combination of solutions suitable for use in cleaning a diaper wearer without causing skin irritations or allergic reactions. Another embodiment may comprise an even milder combination of solutions suitable for a diaper wearer with especially sensitive skin or a lower threshold for allergic reactions.

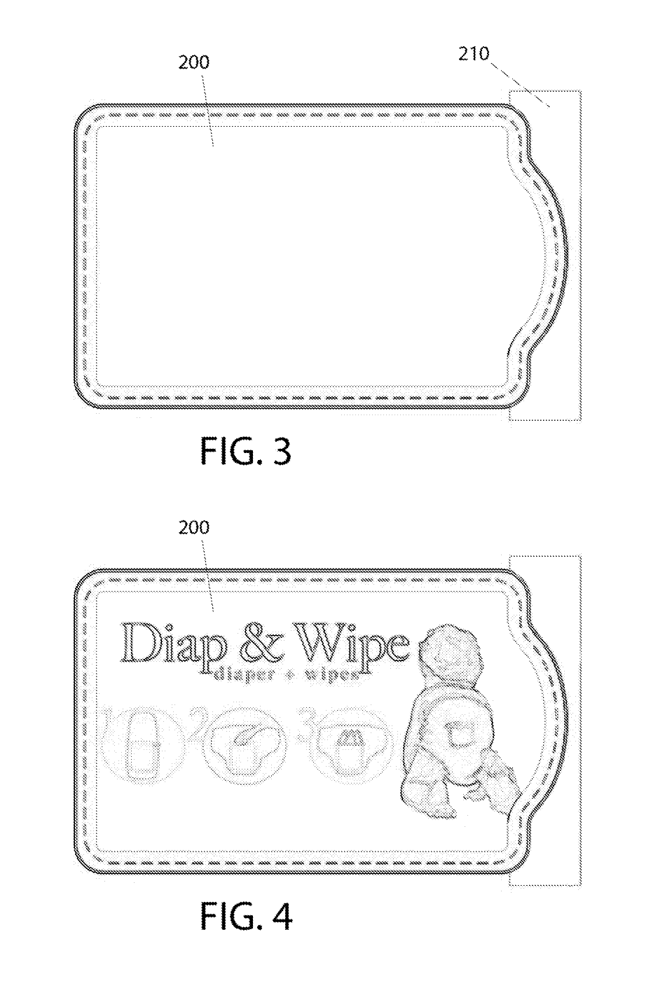

FIG. 3 illustrates a wet wipe pocket layer 200 and a sealable edge 210. The wet wipe pocket layer 200 comprises a plurality of construction layers and any geometric shape suitable for enclosing a plurality of wet wipe packets 100. In a preferred embodiment the wet wipe pocket layer 200 comprises a rectangular shape further comprising a first end, which may be a sealable edge 210. The wet wipe pocket layer 200 may be permanently or removably affixed to an outer face of a wearable absorbent device, and may be affixed to such a wearable absorbent device by any suitable method. In a preferred embodiment the wet wipe pocket layer 200 may be affixed to the outer face of a wearable absorbent device by a combination of temporary adhesives that may allow a user to peel the wet wipe pocket layer 200 away from the wearable absorbent device. The wet wipe pocket layer 200 may also be affixed by sewing, by the use of permanent adhesives, or by heat-welding the wet wipe pocket layer 200 to the wearable absorbent device. In an embodiment where the wet wipe pocket layer 200 is permanently attached to the wearable absorbent device, the sealable edge 210 may be attached by a combination of temporary adhesives so that the pocket may be accessed via the sealable edge 210.

FIG. 4 illustrates a branded wet wipe pocket layer as contemplated by the present invention.

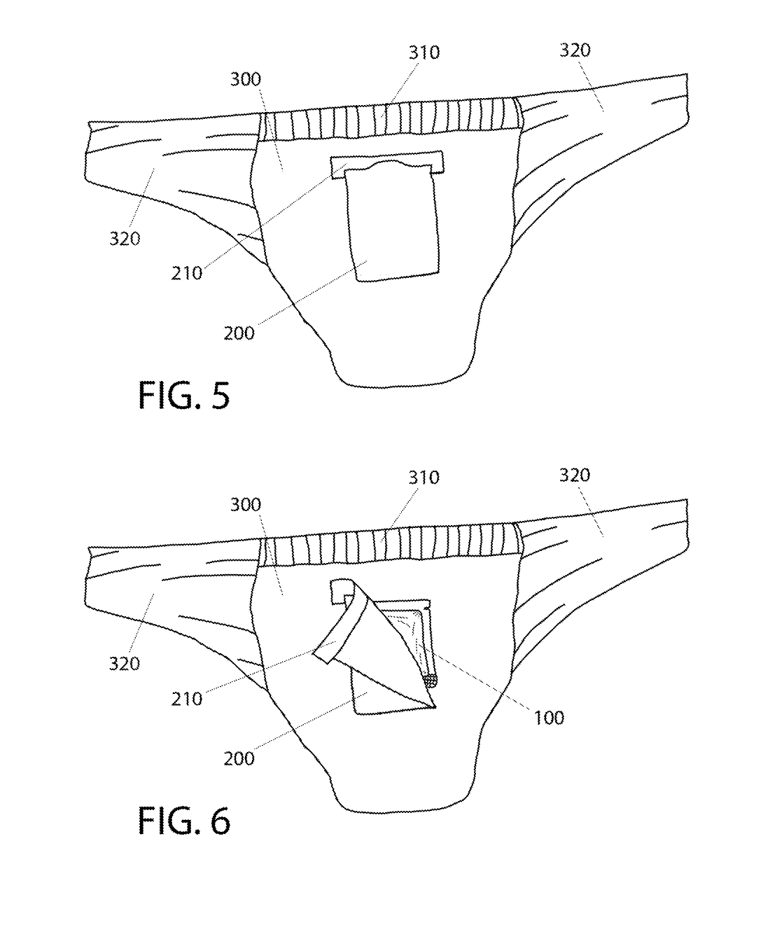

FIGS. 5 and 6 illustrate a first embodiment of a wearable absorbent device and wet wipe combination specifically identifying a diaper body 300, elastic band 310, a plurality of diaper wings 320, and showing the relative positioning of a wet wipe packet 100, wet wipe pocket layer 200, and sealable edge 210. The diaper body 300, as it may be worn by a user, may comprise a front face, a rear face, an upper edge, a lower edge, a first side, and a second side. The upper edge of a diaper body 300 may further comprise an elastic band 310 to facilitate positive engagement of the diaper body 300 against a user's torso. The first side and second side of a diaper body 300 may comprise a plurality of diaper wings 320, which may allow the front face and rear face of a diaper body 300 to positively engage a user's torso and create openings into which the user's legs may be placed. The plurality of diaper wings 320 may be removably attached to a front face of the diaper body 300 and may be permanently attached to a rear face of the diaper body 300, thus facilitating the removal of the diaper body 300 from the user during the changing process.

As contemplated by the present invention, the wet wipe pocket layer 200 may be located on a rear face of the diaper body 300, and a plurality of wet wipe packets 100 may be located in the space formed between the wet wipe pocket layer 200 and the rear face of the diaper body 300. In a preferred embodiment the sealable edge 210 of the wet wipe pocket layer 200 may be oriented towards the upper edge of the diaper body 300 so as to form an upward-opening pocket.

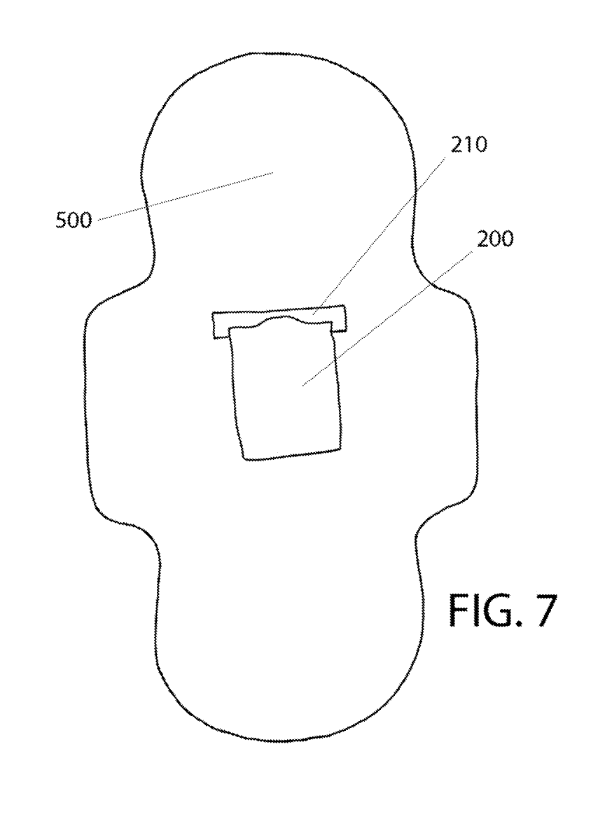

FIG. 7 illustrates a second embodiment of a wearable absorbent device and wet wipe combination specifically identifying a menstrual pad body 500, and showing the relative positioning of a wet wipe pocket layer 200 and sealable edge 210. As contemplated by the present invention, the wet wipe pocket layer 200 may be located on a rear face of the menstrual pad body 500, and a plurality of wet wipe packets 100 may be located in the space formed between the wet wipe pocket layer 200 and the rear face of the menstrual pad body 500.

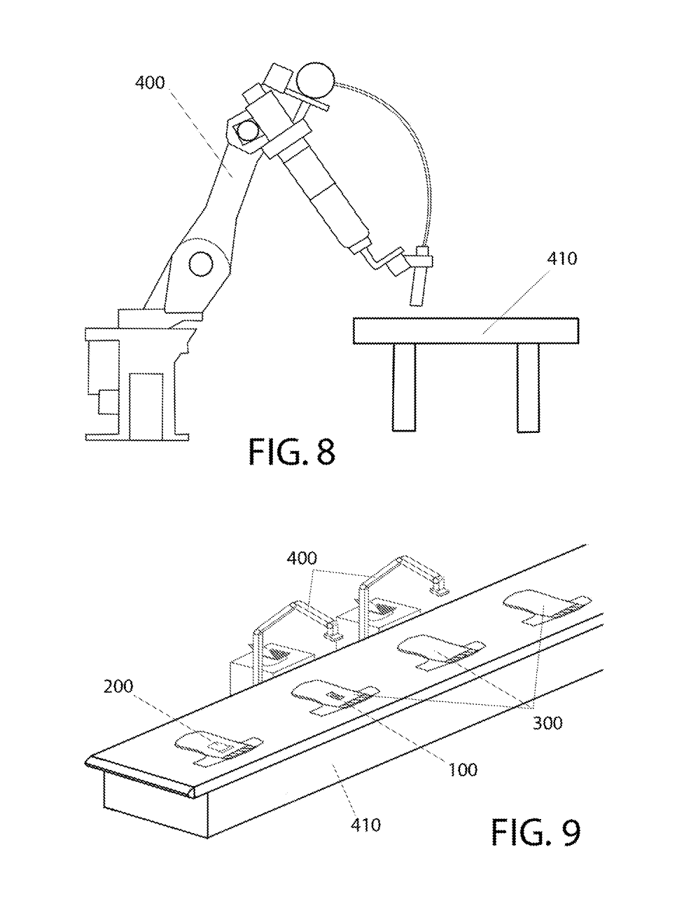

FIG. 8 illustrates a robotic arm 400 for placing wet wipes and installing a wet wipe pocket on a wearable absorbent device. The figure further illustrates a conveyor belt 410. The robotic arm 400 may be any suitable mechanical device, but preferably comprises a plurality of proprietary robotic arms designed to first place a plurality of wet wipes 100 onto a wearable absorbent device, apply an adhesive to a wet wipe pocket layer 200, and then adhere the wet wipe pocket layer onto the wearable absorbent device in such a way as to enclose the plurality of wet wipes 100.

FIG. 9 illustrates a system for placing a plurality of wet wipes 100 and installing a wet wipe pocket layer 200 on a wearable absorbent device. In a preferred embodiment a conveyor belt 410 may be oriented to convey a plurality of wearable absorbent device past a series of robotic arms 400. The series of robotic arms 400 may perform a sequence of actions to assemble the wearable absorbent device and wet wipe combination as contemplated by the present disclosure. Such a sequence may first include placing a plurality of wet wipes 100 onto a wearable absorbent device. The sequence may then involve applying a suitable adhesive to an inside face of a wet wipe pocket layer 200. In an embodiment where the wet wipe pocket layer 200 is attached by sewing or other similar means, this portion of the sequence may be excluded. The sequence may then involve placing the wet wipe pocket layer 200 on to the wearable absorbent device over the plurality of wet wipes 100. The plurality of robotic arms 400 may then attach the wet wipe pocket layer 200 to the wearable absorbent device by any suitable method such as by applying pressure, by applying heat, or by stitching the wet wipe pocket layer 200 to the wearable absorbent device. The plurality of robotic arms 400 may then arrange and package a plurality of wearable absorbent device and wet wipe combinations as part of the sequence.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

I claim:

1. A wearable absorbent device and wet wipe combination, comprising:
a wearable absorbent device;
a wet wipe pocket layer; and
a plurality of wet wipes.

2. The invention of claim 1,
wherein said wearable absorbent device has a front side and a rear side.

3. The invention of claim 2,
wherein said wet wipe pocket layer has an inner face, an outer face, an upper edge, and a lower edge.

4. The invention of claim 3,
wherein said plurality of wet wipes are positioned against said rear side of said wearable absorbent device;
wherein said wet wipe pocket layer is positioned against said rear side of said wearable absorbent device; and
wherein said plurality of wet wipes are located between said wet wipe pocket layer and said rear side of said wearable absorbent device.

5. The invention of claim 4,
wherein said wet wipe pocket layer is attached to said rear side of said wearable absorbent device.

6. The invention of claim 5,
wherein said upper edge of said wet wipe pocket layer is removably attached to said rear side of said wearable absorbent device.

7. The invention of claim 6,
wherein said plurality of wet wipes are individually packaged.

8. The invention of claim 7,
wherein said wearable absorbent device is a disposable diaper.

9. The invention of claim 7,
wherein said wearable absorbent device is a reusable diaper.

10. The invention of claim 7,
wherein said wearable absorbent device is a feminine hygiene pad.

11. A method for making a wearable diaper and wet wipe combination, comprising:
placing a wearable absorbent device on a flat surface;
placing a plurality of wet wipes on said wearable absorbent device;
placing a wet wipe pocket layer on said wearable absorbent device; and
adhering said wet wipe pocket layer to said wearable absorbent device.

12. The method of claim 11,
wherein said placing a wearable absorbent device is performed by a machine;
wherein said placing a plurality of wet wipes is performed by a machine;
wherein said placing a wet wipe pocket layer is performed by a machine; and
wherein said adhering said wet wipe pocket layer to said wearable absorbent device is performed by a machine.

13. The method of claim 12,
wherein said machine is a plurality of robotic arms.
